(19) United States
(12) Patent Application Publication
(10) Pub. No.: US 2005/0240105 A1
(43) Pub. Date: Oct. 27, 2005

(54) Method for reducing electronic artifacts in ultrasound imaging

(76) Inventors: T. Douglas Mast, Cincinnati, OH (US); Waseem Faidi, Clifton Park, NY (US); Inder Raj S. Makin, Loveland, OH (US); Peter G. Barthe, Phoenix, AZ (US); Michael H. Slayton, Tempe, AZ (US); Vadim V. Kouklev, Tempe, AZ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/824,624

(22) Filed: Apr. 14, 2004

(51) Int. Cl. A61B 8/00; A61B 8/12; A61B 8/14

(52) U.S. Cl. 600/443

(57) ABSTRACT

A method for reducing electronic artifacts in ultrasound images of anatomical tissue. At least two calibration signals are received from imaging ultrasound waves that have been reflected from different regions in anatomical tissue. A correction signal is derived from the calibration signals. The correction signal is subtracted from a signal to derive a corrected signal. An image generated from the corrected signal is then displayed. The correction signal may be derived using weighted averaging and may be updated upon receipt of additional signals. The correction signal may be initialized to zero periodically, in response to a change in the system, or at the direction of the operator.

### FIELD OF THE INVENTION

The present invention relates generally to ultrasound, and more particularly, to a method for reducing electronic artifacts in pulse-echo ultrasound imaging.

### BACKGROUND OF THE INVENTION

Ultrasound medical systems and methods include ultrasound imaging of anatomical tissue to identify tissue for medical treatment. Ultrasound may also be used to medically treat and destroy unwanted tissue by heating the tissue. Imaging is done using low-intensity ultrasound waves, while medical treatment is performed with high-intensity ultrasound waves.

Ultrasound waves may be emitted and received by a transducer assembly. The transducer assembly may include a single transducer element, or an array of elements acting together, to image the anatomical tissue and to ultrasonically ablate identified tissue. Transducer elements may employ a concave shape or an acoustic lens to focus or otherwise direct ultrasound energy. Transducer arrays may include planar, concave or convex elements to focus ultrasound energy. Further, array elements may be electronically or mechanically controlled to steer and focus the ultrasound waves emitted by the array to a focal zone to provide three-dimensional medical ultrasound treatment of anatomical tissue. In some treatments the transducer is placed on the surface of the tissue for imaging and/or treatment of areas within the tissue. In other treatments the transducer is surrounded with a balloon that is expanded to contact the surface of the tissue by filling the balloon with a fluid such as a saline solution to provide acoustic coupling between the transducer and the tissue.

Low-intensity ultrasound energy may be applied to unexposed subdermal anatomical tissue for the purpose of examining the tissue. Ultrasound pulses are emitted, and returning echos are measured to determine the characteristics of the unexposed subdermal tissue. Variations in tissue structure and tissue boundaries have varying acoustic impedances, resulting in variations in the strength of ultrasound echos. Time between pulse emission and return of the echo as well as the angle of the echo indicate the location from which the echo is reflected. A common ultrasound imaging technique is known in the art as “B-Mode” wherein either a single ultrasound transducer is articulated or an array of ultrasound transducers is moved or electronically scanned to generate a two-dimensional image of an area of tissue. The generated image is comprised of a plurality of pixels, each pixel corresponding to a portion of the tissue area being examined. The varying strength of the echos is preferably translated to a proportional pixel brightness. A cathode ray tube, computer monitor or liquid crystal display can be used to display a two-dimensional pixellated image of the tissue area being examined.

Ultrasound images frequently include electronic artifacts that appear as non-random noise in the image and that may obscure significant image information. The “main bang”, the electronic pulse that is transmitted through an ultrasound transducer causing transducer vibration and generating the outgoing acoustic wave, causes an artifact associated with interactions of the transmit pulse, transducer elements, and imaging system electronics. Additionally, the main bang causes “ringdown”, or after-ringing of the transducer. The ringdown artifact is caused by a lengthened portion of the electronic signal at the transducer after the main bang, resulting from resonation of the transducer assembly, for example. The main bang and ringdown artifacts appear as high amplitude non-random noise proximate to the transducers that decreases in amplitude with depth. This creates a “dead zone” in the area close to the transducers that may obscure significant echo information. In particular, the dead zone may mask the surface of the anatomical tissue. Since the main bang and ringdown artifacts depend deterministically on the pulse characteristics and the impedance of each channel, the artifacts vary slowly over time and appear nearly constant in imaging performed over a short period of time. These artifacts are especially endemic to devices used for both imaging and medical treatment of tissue because of the conflicting design constraints of high power required for medical treatment and wide bandwidth required for imaging. Other artifacts or undesirable signals include image degradation due to acoustic reflection caused by an intervening structure between the transducer and the tissue, such as a coupling balloon or even an air bubble.

There is a need for a method of eliminating the main bang and ringdown artifacts to display the underlying echo signal data. There is a further need for a method of eliminating electronic artifacts that vary slowly over time in ultrasound images.

### SUMMARY OF THE INVENTION

The present invention provides a method for the reduction of artifacts in ultrasound imaging of anatomical tissue. The method begins with receiving at least two calibration signals of imaging ultrasound waves that have been reflected from different regions in the anatomical tissue. The calibration signals are used to derive a correction signal. The correction signal is then subtracted from a signal of an imaging ultrasound wave to derive a corrected signal. Finally, an image is displayed using the corrected signal.

In another embodiment, the present invention provides for updating the correction signal by receiving at least one additional calibration signal. The additional calibration signal is averaged with the existing correction signal to derive an updated correction signal. The updated correction signal is then subtracted from a signal of an imaging ultrasound wave to derive an updated corrected signal. Finally, an image is displayed using the updated corrected signal.

### BRIEF DESCRIPTION OF THE FIGURES

FIG. 1 is a flow diagram providing an overview of an ultrasound imaging method according to an embodiment of the present invention; and

FIG. 2 is a flow diagram providing an overview of an ultrasound imaging method according to an alternate embodiment of the present invention.

FIG. 3a is illustrative of a B-Mode image generated by ultrasound imaging without the use of the present invention.

FIG. 3b is illustrative of a B-Mode image generated by ultrasound imaging with the use of the present invention.

### DETAILED DESCRIPTION OF THE INVENTION

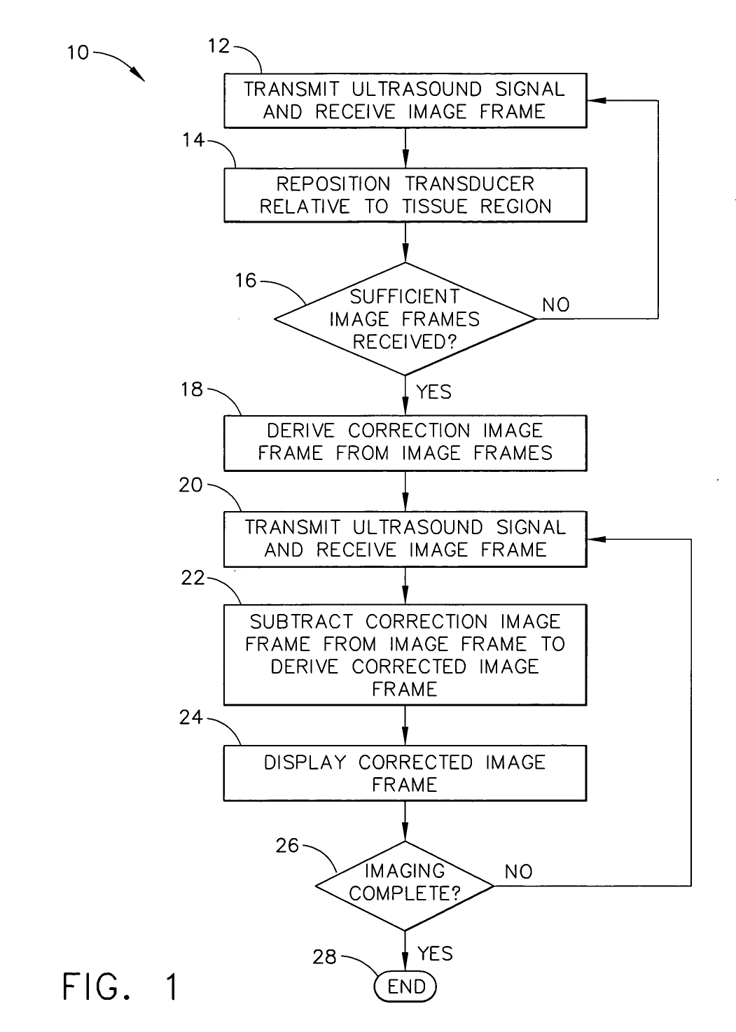

Referring now to the Figures, in which like numerals indicate like elements, FIG. 1 discloses an overview of an ultrasound imaging method with artifact reduction 10 according to an embodiment of the present invention. The method 10 begins at step 12 by transmitting a low-intensity ultrasound signal and receiving reflected echo signals to form an image frame. It is understood that the terminology “image” includes, without limitation, creating an image in a visual form and displayed, for example, on a monitor, screen or display, and creating image data in electronic form that, for example, can be used by a computer without first being displayed in visual form. One possible embodiment of an image frame consists of a two dimensional array, where each element in the array corresponds to a location in the anatomical tissue and has a value corresponding to the RF signal reflected from that location. After the image frame is received in step 12, either the transducer or the tissue is repositioned at step 14 such that a new image frame will encompass a different region of the tissue. The repositioning at step 14 does not require a significant change in location of either the transducer or the tissue. In fact, movement of the tissue due to simple respiration is generally sufficient.

At least two image frames are required to calculate a correction image frame, however, as will be discussed in greater detail below, multiple image frames are required for optimal image correction. If insufficient image frames have been received at step 16 (for example, the number of image frames is less than a predetermined constant), an additional image frame may be received by returning to step 14. If a sufficient number of image frames have been received, a correction image frame is derived from the image frames at step 18.

At step 18 a correction image frame containing only artifacts may be derived from image frames received at step 12. Ultrasound images of anatomical tissue are likely to be dominated by random speckle and there is little or no correlation between images of different tissue regions. In contrast, the electronic artifacts may remain relatively constant throughout repeated imaging over a relatively short period of time. Therefore, when the values of corresponding array elements of the image frames received at step 12 are averaged, array element values in areas of the averaged image frame containing image data from anatomical tissue will approach zero, while array element values of areas of the image frame containing artifacts will tend toward a constant, non-zero value. The most basic method of averaging consists of summing the corresponding array element values of each image frame and dividing by the number of image frames. At least two image frames are required to derive a correction image frame. The accuracy of the correction image frame may be improved by processing larger numbers of image frames. For example, approximately 2540 image frames (comprising about 15 seconds of real-time) have been used to derive the correction image frame. However, this is neither a minimum nor maximum number of image frames necessary to derive the correction image frame.

At step 20, a low-intensity ultrasound signal is transmitted and the reflected echo signals are received to form a new image frame. The correction image frame derived in step 18 is subtracted from this new image frame at step 22 to obtain a corrected image frame. The corrected image frame is used to generate an image to be displayed at step 24. The corrected image frame may include image data previously obscured by artifacts. Alternatively, the correction image frame may be subtracted from any of the image frames received at step 12 to obtain a corrected image frame, and that corrected image frame may be used to generate an image to be displayed at step 24. If imaging is complete at step 26, the method 10 ends at step 28. If additional images are required, the method 10 returns to step 20 to continue imaging of the tissue. The image frames, correction image frame and corrected image frames may be stored electronically, such as in computer, magnetic media and solid-state memory.

In another embodiment of the invention, an updated correction image frame may be recalculated either from all new image frames or from a combination of new image frames and the previously received image frames. In the embodiment depicted in FIG. 1, the correction image frame is determined once and then subtracted from each of the subsequent image frames to derive a corrected image frame for display. Over the course of time, the correction image frame may fail to reflect changes in the artifacts, which could be caused by changes in conditions, such as transducer variations and temperature changes. There are many different methods that may be used to average previous image frames and new image frames to derive a new correction image frame. The most basic method would consist of summing all of the image frames and dividing by the number of image frames. Alternatively, the new correction image frame may be calculated by multiplying the existing correction image frame by the number of image frames used to derive the correction image frame, adding the new image frame and dividing by the number of image frames used to derive the correction image frame plus one, as illustrated in Equation 1:
new correction image frame=((n*correction image frame)+new image frame)/(n+1) Equation 1
where n is equal to the number of image frames used to derive current correction image frame. Additionally, the method could implement a first in first out (FIFO) stack containing a constant number of image frames, in which the first image frame received is the first to be removed from the stack as additional image frames are added and the stack of image frames is used to derive the correction image frame. Numerous methods of averaging data are well known to those skilled in the art, and the present invention is not intended to be limited to the methods discussed herein.

A new correction image frame may be derived either automatically, or at the direction of the operator. The method may be configured to receive new image frames and derive a new correction image frame after a predetermined number of signals have been received or after a predetermined period of time has passed. The method may also derive a new correction image frame upon registering a change in system conditions including, but not limited to, a change to the transducer or a change in temperature. Alternatively, the method may include an operator control to allow the operator to recalculate the correction image frame at any time. In an alternative embodiment, the method may be configured to automatically derive a new correction image frame based upon analysis of the corrected image frames. An increase in amplitude in the portion of the corrected image frame proximate to the transducer may indicate that the method is no longer correctly reducing the artifacts. The method may be configured to calculate the average amplitude for the portion of the corrected image frame proximate to the transducer. This average amplitude may be compared to the average amplitude for the same portion of prior corrected image frames. An increase in this average amplitude may indicate the degradation of the correction image frame and may be used to trigger the derivation of a new correction image frame.

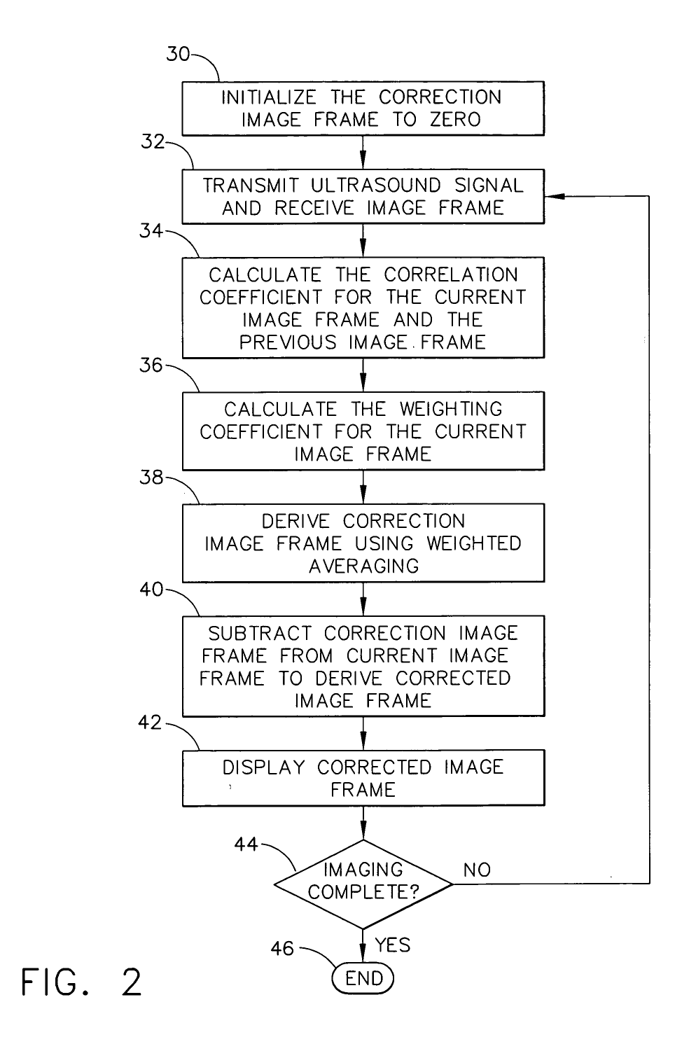

FIG. 2 discloses an alternative embodiment of the present invention using weighted averaging. The method begins at step 30 with the initialization array element values of the correction image frame to zero. At step 32 a low-intensity ultrasound signal is transmitted and reflected echo signals are received to form an image frame. At step 34 Pearson's correlation coefficient is calculated for a region of the image frame and the corresponding region of the previous image frame. The range of values of the correlation coefficient is negative one (−1) to one (1). Identical image frames produce a correlation coefficient of one (1) and inverse image frames produce a correlation coefficient of negative one (−1). The correlation coefficient may be used to determine a weighting coefficient at step 36. The weighting coefficient may be a function of the correlation coefficient such that as the correlation coefficient approaches one (1), the weighting coefficient approaches zero (0) and as the correlation coefficient approaches negative one (−1), the weighting coefficient approaches its maximum value. An example function is depicted in Equation 2:
ε=arccos(r)/π*c Equation 2
where ε is the weighting coefficient, r is the correlation coefficient and c is a user determined constant greater than zero (0) and less than one (1). Therefore, if the new image frame is identical to the prior image frame (for example, the transducer and the tissue are not moved relative to each other), the weighting coefficient for the image frame is zero. At step 38 the weighting coefficient may be used to calculate the updated correction image frame. One possible method of calculating the updated correction image frame consists of taking the sum of the current image frame multiplied by the weighting coefficient and the current correction image frame multiplied by one (1) minus the weighting coefficient, as illustrated in Equation 3:
Updated correction image frame=(1−ε)(current correction image frame)+ε(current image frame) Equation 3

Therefore, when a new image frame is identical to the previous image frame, the weighting coefficient is zero (0) and the new image frame will not affect the derivation of the updated correction image frame. The updated correction image frame is subtracted from the image frame in step 40 to obtain a corrected image frame. The corrected image frame is used to generate an image to be displayed at step 42. The method may return to step 32 and receive an additional image frame, which will then be used to derive an updated correction image frame. Further embodiments could include the ability to dynamically stop and start updating the correction image frame as well as the ability to reinitialize the correction image frame to zero.

In another embodiment of the invention, the operator may be able to view the uncorrected image frame or the correction image frame, as well as the corrected image frame. An operator control may be added to allow the operator to switch between displays of the corrected image frame, the uncorrected image frame and the correction image frame. Additional display screens may include combinations of the displays, or all three displays on the screen at the same time.

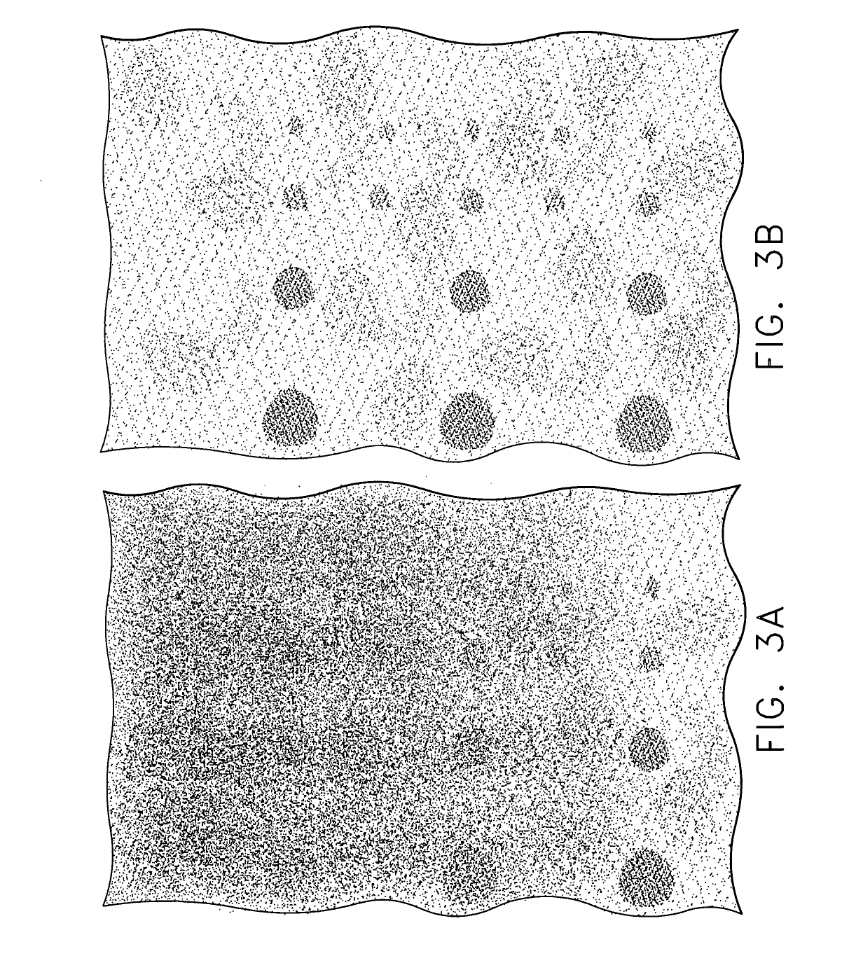

FIGS. 3a and 3b illustrate B-Mode images of the same test object generated by ultrasound imaging. Each pixel in the figures represents a location in the test object. The varying strength of the echos reflected from the different locations is translated to a proportional pixel brightness. The pixels at the top of each of the images in FIGS. 3a and 3b represent locations proximate to the transducer array. Pixels closer to the bottom of the images represent locations at a greater depth within the test object and therefore farther from the transducer array. FIG. 3a illustrates a B-Mode image generated without the use of the present invention. Image data at the top of the image, proximate to the transducer, is obscured by the main bang and ringdown artifacts. The artifacts appear as high amplitude noise that decreases in amplitude with depth. FIG. 3b illustrates a B-Mode image generated with the use of the present invention. The artifacts have been removed, allowing the user to see the underlying image data.

While the present invention has been illustrated by description of several embodiments, it is not the intention of the applicant to restrict or limit the spirit and scope of the appended claims to such detail. Numerous other variations, changes, and substitutions will occur to those skilled in the art without departing from the scope of the invention. For instance, the method of the present invention has been illustrated in relation to ultrasound imaging, but it will be understood the present invention has applicability in other types of imaging as well. Moreover, the structure of each element associated with the present invention can be alternatively described as a means for providing the function performed by the element. It will be understood that the foregoing description is provided by way of example, and that other modifications may occur to those skilled in the art without departing from the scope and spirit of the appended claims.

1. A method for reducing artifacts in ultrasound imaging of anatomical tissue, comprising the steps of:
a) receiving at least two calibration signals of imaging ultrasound waves that have been reflected from different regions in the anatomical tissue;
b) deriving a correction signal from the calibration signals;
c) subtracting the correction signal from a signal of an imaging ultrasound wave to derive a corrected signal; and
d) displaying an image using the corrected signal.

2. The method of claim 1, wherein the correction signal is derived by averaging of the calibration signals.

3. The method of claim 2, wherein the correction signal is derived by weighted averaging of the calibration signals.

4. The method of claim 3, wherein the weighted average assigns a higher weight to the calibration signals that are not correlated to a prior calibration signal.

5. The method of claim 1, further including the step of displaying an image using the uncorrected signal.

6. The method of claim 1, wherein the artifacts to be reduced are caused by main bang and ringdown signals.

7. The method of claim 1, wherein the artifacts to be reduced are caused by acoustic reflection from an intervening structure.

8. The method of claim 1, wherein:
a) the correction signal is updated by receiving at least one additional calibration signal and averaging the additional calibration signal and the calibration signals previously received;
b) the updated correction signal is subtracted from a signal of an imaging ultrasound wave to derive an updated corrected signal; and
c) an image is displayed using the updated corrected signal.

9. The method of claim 1, wherein the anatomical tissue is moved such that the calibration signals are reflected from different regions.

10. The method of claim 9, wherein the movement of the anatomical tissue is accomplished by respiration.

11. The method of claim 1, wherein a transducer is moved such that the calibration signals are reflected from different regions.

12. The method of claim 1, wherein the step of receiving the calibration signals is conducted using at least ten calibration signals.

13. The method of claim 1, wherein the correction signal is set to zero from time to time and a new correction signal is obtained by receiving additional calibration signals and averaging the additional calibration signals to derive a new correction signal, subtracting the new correction signal from a signal of an imaging ultrasound wave to derive a corrected signal and displaying an image using the corrected signal.

14. The method of claim 13, wherein the correction signal is set to zero at regular intervals.

15. The method of claim 13, wherein an operator may elect to set the correction signal to zero.

16. The method of claim 13, wherein the correction signal is set to zero when there is a change in system conditions.

17. The method of claim 13, wherein the correction signal is set to zero based upon an analysis of the corrected signal.

18. The method of claim 13, wherein the correction signal is set to zero based at least in part upon an average amplitude of at least a portion of the corrected signal.

19. The method of claim 13, wherein the correction signal is set to zero when there is a change in temperature of a transducer.

20. The method of claim 1, further including the step of displaying an image using the correction signal.

21. A method for reducing artifacts in ultrasound imaging of anatomical tissue, comprising the steps of:
a) receiving at least two calibration signals of imaging ultrasound waves that have been reflected from different regions in the anatomical tissue;
b) deriving a correction signal by weighted averaging of the calibration signals, where the weighted average assigns a higher weight to calibration signals that are not correlated to a prior calibration signals;
c) subtracting the correction signal from a signal of an imaging ultrasound wave to derive a corrected signal;
d) displaying an image using the corrected signal;
e) updating the correction signal by receiving at least one additional calibration signal and averaging the additional calibration signal and the calibration signals previously received;
f) subtracting the updated correction signal from a signal of an imaging ultrasound wave to derive an updated corrected signal; and
g) displaying an image using the updated corrected signal.

22. The method of claim 21, further including the step of displaying an image using the uncorrected signal.

23. The method of claim 21, further including the step of displaying an image using the correction signal.

24. The method of claim 21, wherein the correction signal is set to zero from time to time and a new correction signal is obtained by receiving additional calibration signals and averaging the additional calibration signals to derive a new correction signal, subtracting the new correction signal from a signal of an imaging ultrasound wave to derive a corrected signal and displaying an image using the corrected signal.
